(19) United States
(12) Patent Application Publication
(10) Pub. No.: US 2008/0152695 A1
(43) Pub. Date: Jun. 26, 2008

(54) ORAL/BUCCAL TRANSMUCOSAL DELIVERY METHODS FOR ELECTROLYTE COMPOSITIONS INCLUDING XYLITOL

(76) Inventors: Richard T. Clark, Tulsa, OK (US); Maurice E. Durschlag, Charlotte, NC (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/763,023

(22) Filed: Jun. 14, 2007

Related U.S. Application Data
(60) Provisional application No. 60/813,384, filed on Jun. 14, 2006.

(51) Int. Cl. A61K 31/7004; A61K 9/00; A61P 43/00

(52) U.S. Cl. 424/439; 514/25

(57) ABSTRACT

The present disclosure is an oral/buccal transmucosal delivery method which includes, generally, an edible thin film strip including a predetermined volume of xylitol. The edible thin film strip of the present disclosure is preferably comprised of potassium chloride; trisodium citrate; sodium chloride; glucose, and; xylitol.

### BACKGROUND

1. Field of the Invention

The present invention relates, generally to various oral/buccal transmucosal systems for delivering medicaments or other such substances to mammal and/or human bodies.

2. Description of the Related Art

Therapeutics can be effectively delivered across the mucous membrane. Transmucosal delivery is particularly attractive because these membranes are very thin and permeable. Such properties allow for the rapid uptake of a drug (substance) into the body. This efficient uptake allows drugs (substances) to bypass some of the body's natural defenses and enhances the effect of the therapeutic. Transmucosal delivery systems offer several benefits over other methods of delivery including:

Direct Absorption: Absorption through the mucous membrane leads directly to the circulatory system. This allows drugs (substances) to bypass the gastrointestinal tract as well as first pass liver metabolism. This is important for biological therapeutics.
Rapid Onset Drugs: (substances) directly enter the circulatory system, which allows the therapeutic to be rapidly transported to the site of need. The faster the drug/substance reached its target area, the faster it can begin to elicit its desired effect.
Lower Dosage The avoidance of the gastrointestinal tract and first pass metabolism means that much less of the drug can be administered to achieve the same effect, allowing for lower dosages to be administered and fewer side effects.
Transmucosal drug delivery is generally classified into three systems:

### Nasal Transmucosal

Products in this category include nasal sprays, pumps, and gels. The majority of the drugs delivered to the nasal passage are anti-inflammatories.

### Oral/Buccal Transmucosals

These systems make use of saliva to release the therapeutic. Products include mucoadhesives, quick-dissolve strips or other quick dissolve delivery systems, reservoir pouches or packets, and solid lozenge formulations.

### Vaginal or Urethral Suppositories

Delivery systems in this category are designated to be absorbed directly by the vaginal or penile capillary beds.

A viscous polysaccharide matrix designed to trap foreign particles that may enter the system coats the mouth, nasal passage, vagina and urethra. This is a defense, which prevents damage to delicate tissues and capillary beds which lie directly underneath the epithelium. Though the mucous membrane protects the body from foreign matter and pathogens, the area is much more permeable than mucous membrane. This permeability allows drugs delivered to the mucous membrane to quickly enter into circulation.

Transmucosal delivery of medicaments and the like offers advantages over oral delivery when negative issues relating to the gastro-intestinal tract, the stomach, substance digestion and absorption, swallowing, protocol compliance, and substance effectiveness as well as other issues with respect to gastrointestinal metabolism are considered.

Xylitol is a sweetening agent that, in its pure form, is a white crystalline substance that looks and tastes like sugar. Xylitol as a small dietary addition has demonstrated a dramatic reduction in new tooth decay, along with arrest and even some reversal of existing decay. Xylitol provides additional protection that enhances all existing prevention methods. In the amounts needed to prevent tooth decay, such as approximately 15 grams per day, xylitol is safe for all persons. It has been determined that xylitol is most effective when used multiple times daily, preferably three (3) and most preferably five (5) times every day. Thus, a need exists for a oral/buccal transmucosal delivery method for the delivery of xylitol as a preventative for tooth decay.

Liquid electrolyte drinks, such as sports drinks are well known and the benefits of replenishing electrolytes during periods of intense physical exertion are well documented. However, the delay in absorption of electrolytes from the ingestion of liquids can reduce their effectiveness, particularly during intense exercise. A need, therefore exists for an oral/buccal transmucosal delivery method for the delivery of xylitol coupled with the delivery of electrolytes.

### SUMMARY

The present disclosure describes an oral/buccal transmucosal delivery method which includes, generally, an edible thin film strip including a predetermined volume of xylitol. Such oral/buccal transmucosal methods include quick dissolve strips, thin-film composites, powders, gels, sprays, time release lozenge or reservoir packets, and others. An edible thin film electrolyte strip of the present disclosure is preferably comprised of potassium chloride; trisodium citrate; sodium chloride; glucose, and; xylitol.

The preferred method of manufacture of the edible thin film strip of the present disclosure includes the basic steps of: obtaining a slurry of compounded potassium chloride, trisodium citrate, sodium chloride, glucose and xylitol; extruding a thin film of slurry onto a release liner, and; drying the extruded slurry. The method may include the additional steps of removing the dried extruded slurry from the release liner; cutting the dried extruded slurry into predetermined sizes, and; packaging the dried and cut extruded slurry. The dried and cut extruded slurry is preferably packaged in a cassette.

Suitable oral/buccal transmucosal systems for the present disclosure include the 3M Cydot™ System offered in several configurations including matrix and reservoir designs; Zengen Inc.'s “oral strip bilayer system” which is being used in Chloraseptic Relief Strips™. A similar system like that utilized in Pfizer Inc.'s Cool Mint Listerine PocketPacks™ Oral Care Strips would also suffice. Yet another system could be a “tea bag” device similar to a Skoal Bandit® product.

The oral/buccal transmucosal method of the present disclosure may also include various vitamins and minerals generally known to be provided as nutritional supplements which may include Iron, Sodium, Calcium, Magnesium, Carbohydrates, Proteins, Sugars (Glucose), Zinc, Molybdenum, Copper, Manganese, Chlorides, Bicarbonate and Carbonate, Aluminum, Arsenic, Bromine, Cadmium, Chromium, Chlorine, Cobalt, Fluorine, Iodine, Manganese, Molybdenum Nickel, Phosphorus, Selenium, Silicon, Vanadium, Zinc, Amino Acids, Vitamin A, Vitamin D, Vitamin E, Vitamin K, Vitamin C, Vitamin B complex, Thiamine (Vitamin B1), Riboflavin (Vitamin B2), Niacin (Vitamin B3), Pyridoxine (Vitamin B6), Biotin, Pantothenic Acid and Pantethine, Folic Acid, Vitamin B12, “Unofficial” B Vitamins including Choline and Inositol, Vitamin P (bioflavonoids), and/or other vital nutrients, in addition to various bomeopathic/alternative substances.

A highly suitable application for the method of the present disclosure is for military uses. It is known that soldiers are often required to serve in areas and/or conditions where regular oral hygiene is not practical or even possible. Use of the oral/buccal transmucosal delivery method of the present disclosure would allow such soldiers to intake the xylitol in amounts which are calculated to assist in the prevention of tooth decay. In addition, the system of the present invention coupled with electrolytes is highly suitable for use by military personnel when undergoing physical stress or exertion.

It is an object of the present disclosure to provide an edible thin film strip which includes xylitol.

It is a further object of the present disclosure to provide an edible thin film strip including xylitol and electrolytes.

Additional objects and advantages of the present invention will become apparent to those skilled in the art to which this invention relates from the following detailed description of the preferred embodiment and the claims

### DETAILED DESCRIPTION OF THE PREFERRED EMBODIMENTS

Before explaining the present invention in detail, it is important to understand that the invention is not limited in its application to the details of the embodiments and steps described herein. The invention is capable of other embodiments and of being practiced or carried out in a variety of ways. It is to be understood that the phraseology and terminology employed herein is for the purpose of description and not of limitation.

The present invention includes an oral/buccal transmucosal delivery method for electrolyte compositions including xylitol. Specifically, the invention is a method of delivery for an electrolyte composition including xylitol as well as a thin film electrolytic strip containing xylitol.

Oral/buccal transmucosal delivery methods provide for the rapid introduction of active ingredients, such as electrolytes and the like into the bloodstream.

Liquid electrolyte drinks, such as sports drinks are well known and the benefits of replenishing electrolytes during periods of intense physical exertion are well documented. However, the delay in absorption of electrolytes from the ingestion of liquids can reduce their effectiveness, particularly during intense exercise. In contrast, delivery of electrolyte compositions through oral/buccal transmucosal methods according to the present invention delivers the electrolytes to the bloodstream much faster.

The Cations and Anions of fluid electrolyte composition are never static, but are proportionately balanced within the compensatory rates of metabolic activity both intracellularly and extracellularly. Pivotal losses of calcium and magnesium from muscle exhaustion, fluid dehydration from sweat loss, depletion of extracellular cation stores of sodium or intracellular cation stores of potassium are significant factors staged for muscle failure, i.e., a cramp event. Strong evidence exists for the role of electrolyte depletion associated with muscle spasms, cramps, and seizures. In addition, depletion of muscle glycogen, fluid overhydration, and the lack of vitamin substrates with enzymatic influence on fuel selection are also presently considered suspects.

Xylitol is a sweetening agent that, in its pure form, is a white crystalline substance that looks and tastes like sugar. Xylitol is classified broadly as a carbohydrate and more narrowly as a polyol. Due to the fact that xylitol is only slowly absorbed and partially utilized, a reduced calorie claim is allowed when compared to other carbohydrates. In the United States, xylitol is approved as a food additive in unlimited quantity for foods with special dietary purposes.

Xylitol has been known to reduce tooth decay rates in both high risk groups, such as groups with poor oral hygiene, and low risk groups, such as those employing all current prevention recommendations. Sugar-free chewing gums and candies made with xylitol as the principal sweetener have already received official endorsements from six (6) national dental associations. It is contemplated that an electrolyte strip, including xylitol, will be particularly suitable for military use and other applications where traditional forms of oral hygiene may not be practical in the field.

Xylitol as a small dietary addition has demonstrated a dramatic reduction in new tooth decay, along with arrest and even some reversal of existing decay. Xylitol provides additional protection that enhances all existing prevention methods. In the amounts needed to prevent tooth decay, such as approximately 15 grams per day, xylitol is safe for all persons. It has been determined that xylitol is most effective when used multiple times daily, preferably three (3) and most preferably five (5) times every day.

As a result, the addition of xylitol to an oral/buccal transmucosal delivery method for use multiple times daily is helpful in the reduction of tooth decay.

The present disclosure relates to transmucosal methods and products for supplying xylitol in addition to replenishing electrolytes and additional components to a subject involved in strenuous exercise which avoids many of the limitations associated with sports drinks or other orally ingested supplements.

The invention in one aspect is a quick-dissolve strip (or reservoir pouch/packet) for administering xylitol in combination with electrolytes and/or nutrients utilized during exercise and other periods of high energy exertion. The xylitol and nutrients are delivered from the quick-dissolve strip directly to the blood stream where they can supply the necessary energy or maintenance of homeostatic conditions in the body. There is no need for the nutrients to pass through the gastrointestinal tract where absorption would be a limiting factor.

The delivery of an athletic supplement using a quick-dissolve strip offers several advantages over traditional delivery methods. For instance, the quick-dissolve strip avoids gastrointestinal metabolism of the athletic supplement, reduces first pass effects and may if desirable provide a longer course of release of the components of the athletic supplement than traditional methods such as the use of sports drinks or other orally ingested nutritional supplements. The quick-dissolve strip may include many different concentrations of the components of the athletic supplement.

The quick-dissolve strip may be any type of conventional quick-dissolve strip, such as a transmucosal quick-dissolve strip, a sublingual quick-dissolve strip, or a buccal quick-dissolve strip. In a preferred embodiment the quick-dissolve strip includes a permeation enhancing amount of at least one mucous membrane permeation enhancer.

The transmucosal quick-dissolve strip may be of any shape, such as oblong, square, round, rectangular, etc. The transmucosal quick-dissolve strip may also have a variety of sizes.

The quick-dissolve strip of the present disclosure provides xylitol in combination with all of the nutritional, carbohydrate, and energy requirements of an athlete under conditions of physical stress without causing gastrointestinal disturbances.

In a preferred embodiment, an oral/buccal transmucosal delivery method would include an edible thin film strip for insertion in the oral cavity. The strip is preferably 0.875″ wide×1.25″ long.

The product is defined as a cast thin film cut into strips 0.875″ wide×1.25″ long. The strips are formulated to dissolve instantly when placed on the tongue. The inventive strip formulas will be manufactured in an OTC licensed factory under strict cGMP. The final product will be placed into a primary package which can be referred to as a cassette. The cassette is made of plastic consisting of a top and bottom. The top portion will have a flip top. The primary cassette “dispenser” will then be placed on a cardboard card backed with aluminum and sealed in place on the card using a PVC blister.

Both strip formulas will contain a thin film forming polymer such as pectin.

In a preferred method of manufacture of the strip, a premixed slurry is compounded in a stainless steel mixing vessel under heat and vacuum. The slurry is then pumped to a holding tank where the product is transferred via a gear to the thin film forming line. The slurry is then extruded evenly through a slot die onto an 18″ wide polyester release liner. The slurry is then extruded onto a wet coated release liner that is then conveyed into an electric reflow tandem heated oven/dryer unit. A computer programmed dryer, consisting of multiple zones that temper and then dry the film thus removing moisture to a specified level is employed. The drying line parameters include preprogrammed instructions which control processing parameters like line speed, air volume, velocity, RH and temperature. The film is then cut into 1.25″ spools on 3″ cores and stored for a specified period prior to converting and packaging.

The converting/packaging process beings with a cured-coated spool that is unwound and slit to ⅞″ wide strips. As the roll is unwound, it is delaminated from the polyester release liner. The film is driven to the cutting station for final cutting into thirty-two (32) individual 0.875″ lengths and then placed into the lower (bottom) of the cassette. Each cassette receives thirty-two (32) strips before moving to the cassette lid feeding station where the lid can be manually or automatically placed onto the bottom of the cassette. The filled and closed cassette is delivered to a labeling unit to apply front and back labels. Final labeled units are then placed into an intermittent motion thermoform machine that heat seals a formed clear web to a foil based lid stock. The clear barrier thermoform web and film lid stock protects the product against moisture and flavor loss. The cassette is now ready for secondary packaging, blister carding and display pack out.

By way of example, an electrolyte strip according to the present invention may include:
1 part Potassium Chloride
1.93 parts Trisodium Citrate
2.33 parts Sodium Chloride
13.33 parts Glucose.

In addition, each strip preferably includes approximately 2 milligrams of xylitol. The xylitol provides sweetening properties to the strip and also the known tooth decay prevention properties.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are compassed within the spirit of this invention.

What is claimed is:

1. An oral/buccal transmucosal delivery method, comprising:
an edible thin film strip including a predetermined volume of xylitol.

2. An electrolyte strip, comprising:
potassium chloride;
trisodium citrate;
sodium chloride;
glucose, and;
xylitol.

3. The electrolyte strip of claim 1, comprising
approximately 1 part potassium chloride;
approximately 1.93 parts trisodium citrate;
approximately 2.33 parts sodium chloride;
approximately 13.33 parts glucose, and;
approximately 2 milligrams of xylitol.

4. A method of producing an edible thin film strip, comprising:
obtaining a slurry of compounded potassium chloride, trisodium citrate, sodium chloride, glucose and xylitol;
extruding a thin film of said slurry onto a release liner;
drying said extruded slurry.

5. The method of claim 4 further including:
removing said dried extruded slurry from said release liner;
cutting said dried extruded slurry into predetermined sizes.

6. The method of claim 5 further comprising:
packaging said dried and cut extruded slurry.

7. The method of claim 6 wherein said dried and cut extruded slurry is packaged in a cassette.

8. The method of claim 5 wherein said dried extruded slurry is cut in strips measuring approximately ⅞″ by 0.875″.

9. The method of claim 8 further comprising:
packaging said dried and cut extruded slurry.

10. The method of claim 9 wherein said dried and cut extruded slurry is packaged in a cassette.

11. The method of claim 10 wherein approximately 32 strips are packaged in a cassette.

12. The method of claim 4 wherein said slurry comprises:
approximately 1 part potassium chloride;
approximately 1.93 parts trisodium citrate;
approximately 2.33 parts sodium chloride;
approximately 13.33 parts glucose, and;
approximately 2 milligrams of xylitol.
